(19) United States
(12) Patent Application Publication
(10) Pub. No.: US 2005/0170353 A1
(43) Pub. Date: Aug. 4, 2005

(54) Gene examination method for judging the onset risk of glaucoma

(75) Inventors: Kaoru Asano, Hyogo (JP); Takayuki Takahata, Hyogo (JP); Shigehiro Numada, Hyogo (JP); Akinori Masago, Hyogo (JP); Yasuhiro Kouchi, Hyogo (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Hyogo (JP)

(21) Appl. No.: 10/509,595

(22) PCT Filed: Mar. 19, 2003
(86) PCT No.: PCT/JP03/03307

(30) Foreign Application Priority Data
Mar. 29, 2002 (JP) 2002-93443

(51) Int. Cl. C12Q 1/68

(52) U.S. Cl. 435/6

(57) ABSTRACT

To provide a gene examination method for predicting the onset risk of glaucoma based on the relation between a glaucoma-associated gene and the onset of glaucoma, the onset of glaucoma in future is predicted by using as an indication mutations in the gene region involving the code region and/or the upstream region of a glaucoma-associated gene. In the base sequence represented by SEQ ID NO:1, mutation(s) in at least one of the following positions are detected, i.e., the 194-, 199-, 324-, 1051-, 1084-, 1627-, 1685-, 1756-, 1853-, 2830-, 3371-, 4037- and 4364-positions.

### FIELD OF THE INVENTION

The present invention relates to a method for examining a glaucoma-related gene in the field of a clinical examination, and an examination method employing a variation in this gene as an index for the purpose of predicting any risk of developing glaucoma. For example, a method for examining a gene by detecting any abnormality in a myocilin (hereinafter referred to as MYOC) gene known as a glaucoma gene followed by diagnosing the glaucoma using as an index the abnormality thus detected, i.e., a variation in a base in a particular site in the gene, especially, an examination method for predicting any possibility of developing in future for a certain individual, is contemplated.

### BACKGROUND OF THE INVENTION

Glaucoma is a disease involving a difficulty in excreting an aqueous humor in an eye, which leads to an increased ocular tension, resulting in a reduced ocular function. If being left untreated, it reduces the visual field and the sight, resulting in a blindness. A normal ocular tension, however, may also cause an impairment of an optic nerve.

Glaucoma is classified into any one of the five pathological types, namely, primary open-angle glaucoma (POAG), normal tension glaucoma (NTG), primary angle-closure glaucoma (PACG), congenital glaucoma and secondary glaucoma, and 20% of the glaucoma is considered to be congenital. Among these types, the most frequent one is POAG. The epidemiological investigation conducted over the period from 1988 to 1989 by Japan Ophthalmologists Association, reported that 3.56% of the population of 40 years old or older consists of the glaucoma patient.

A major risk factor of glaucoma is a familial history, and the development of glaucoma is suggested positively to be associated with a gene. In the U.S. Pat. No. 5,789,169 to Nguyen et al filed on May 17, 1996, a gene encoding a TIGR (trabecular reticulum-induced glucocorticoid responsive) protein is disclosed as a glaucoma-related gene. The TIGR gene is referred to also as MYOC gene. The U.S. Pat. No. 5,789,169 to Nguyen et al also disclosed the cDNA sequence of this protein, the protein itself, a molecule binding thereto and a nucleic acid molecule encoding this binding molecule, and provided improved methods and reagents for diagnosing glaucoma and related diseases, as well as for diagnosing cardiovascular diseases, immune diseases or other diseases or conditions affecting the expression or activity of this protein. On the other hand, a method for diagnosing glaucoma in an individual by detecting a mutation in a CYP1B1 gene among the glaucoma-related genes followed by using the presence of this mutation as an index of glaucoma is disclosed (JP-W-2001-512969). Nevertheless, any of the publications described above was not successful in disclosing a means for predicting any risk of developing glaucoma in future, although it focused on the relationship between the glaucoma-related genes and the glaucoma.

On the other hand, WO01/88120A1 discloses a method for detecting a variation in the gene at position-153 in an MYOC gene promoter region represented in the sequence listing in this publication, and describes that the method can be used in screening for glaucoma in a patient whose familial genetic predisposition is problematic or who is suspected to be a carrier not developed. Nevertheless, the variation just at position-153 as the only one position is just focused on here and used as an index.

Since glaucoma is a latent disease, a further excellent method for enabling an early diagnosis or an effective prediction of possibility for developing glaucoma is desired in order to ensure the prevention or amelioration prior to a serious impairment of an optic nerve.

### DESCRIPTION OF THE INVENTION

(Problem to be Solved)

An early diagnosis and early treatment of glaucoma is possible efficiently if it is possible to identify an individual who has a genetic risk factor of the glaucoma and who is at an elevated risk of developing in future and to perform a glaucoma examination in this individual intensively. Under such a circumstance, an objective of the invention is to provide a method for examining a gene for the purpose of effectively predicting a risk of developing glaucoma based on the relationship between a glaucoma-related gene and the development of glaucoma.

(Means for Solving Problem)

We focused on the involvement of a gene variation in a development of glaucoma and analyzed the gene sequence in the upstream region and the coding region of the glaucoma-responsible gene in glaucoma patients and non-patients, and finally discovered as a result of keen examination that there is a genetic polymorphism in this gene whose frequency is different between the patient group and the non-patient group. Furthermore, we discovered that the glaucoma incidence differs in a statistically significant manner when compared with the incidence in an ordinary population on the basis of the presence or absence of this genetic polymorphism and completed the present invention.

Thus, the invention consists of:
1. a method for examining a gene comprising detecting a variation in bases in at least two or more positions within a gene region containing a glaucoma-related gene coding region and/or an upstream region and predicting any development of glaucoma in future using said variation as an index;
2. the method according to the above-mentioned 1 wherein the glaucoma-related gene is a myocilin (MYOC) gene;
3. the method according to the above-mentioned 1 or 2 wherein the gene region is the nucleic-acid base sequence represented by SEQ ID. No: 1;
4. the method according to any one of the above-mentioned 1 to 3 wherein the nucleic-acid base variation is a substitution, deletion and/or insertion;
5. a method according to the above-mentioned 3 which detects any of the group consisting of, in the nucleic-acid base sequence represented by SEQ ID No: 1, the C-to-A substitution at position 194; the A-to-C substitution at position 199; the G-to-A substitution at position 324; the C-to-T substitution at position 1051; the C-to-T substitution at position 1084; the T-to-C substitution at position 1627; the T-to-C substitution at position 1685; the C-to-T substitution at position 1756; the G-to-C substitution at position 1853; the G-to-A substitution at position 2830; the A-to-G substitution at position 3371; the G-to-A substitution at position 4037; and the G-to-A substitution at position 4346;
6. a method according to the above-mentioned 3 which detects at least two or more simultaneous substitutions selected from the group consisting of, in the nucleic-acid base sequence represented by SEQ ID No: 1, the C-to-A substitution at position 194; the C-to-T substitution at position 1084; the T-to-C substitution at position 1627; the G-to-A substitution at position 4037; and the G-to-A substitution at position 4346;
7. the method according to the above-mentioned 3 which detects at least two or more simultaneous substitutions selected from the group consisting of, in the nucleic-acid base sequence represented by SEQ ID No: 1, the C-to-T substitution at position 1051; the T-to-C substitution at position 1685; the C-to-T substitution at position 1756; and the G-to-C substitution at position 1853;
8. a method for examining a gene comprising detecting at least one substitution from the group consisting of, in the nucleic-acid base sequence represented by SEQ ID No: 1, the A-to-C substitution at position 199; the G-to-A substitution at position 324; the C-to-T substitution at position 1051; the C-to-T substitution at position 1084; the T-to-C substitution at position 1627; the T-to-C substitution at position 1685; the C-to-T substitution at position 1756; the G-to-C substitution at position 1853; the G-to-A substitution at position 2830; and the A-to-G substitution at position 3371 and predicting any future development of glaucoma using said variation as an index;
9. the method according to any one of the above-mentioned 1 to 8 wherein the glaucoma is primary open-angle glaucoma and/or normal tension glaucoma;
10. the method according to any one of the above-mentioned 1 to 9 wherein the variation is detected using an oligonucleotide capable of specifically forming a hybrid with a part of a gene region containing a glaucoma-related gene coding region and/or an upstream region;
11. a primer function-possessing oligonucleotide wherein the oligonucleotide, which is capable of specifically forming a hybrid with a part of a gene region containing a glaucoma-related gene coding region and/or an upstream region, is at least one or more selected from the group consisting of:
1) an oligonucleotide consisting of a nucleic-acid base sequence represented by any of SEQ ID Nos; 2 to 27;
2) a strand complementary with the oligonucleotide according to the above-mentioned 1);
3) an oligonucleotide capable of hybridizing under a stringent condition with the oligonucleotide according to the above-mentioned 1) or 2);
4) an oligonucleotide having a homology of about 60% with the oligonucleotide according to any one of the above-mentioned 1) to 3);
5) an oligonucleotide having a nucleic-acid base sequence whose 1 or more base of oligonucleotides according to the above-mentioned 1) to 4) was subjected to a variation such as a substitution, deletion, insertion or addition;
12. a method according to any one of the above-mentioned 1 to 9 comprising performing a nucleic acid amplification process using at least one oligonucleotide selected from the oligonucleotides according to the above-mentioned 11;
13. an examination reagent or examination reagent kit comprising a reagent used in the examination method according to any one of the above-mentioned 1 to 10 or the above-mentioned 12.

### BRIEF DESCRIPTION OF THE DRAWINGS

FIG. 1 shows the structure of an MYOC gene and the relative position of the primer (Example 1).

FIG. 2 shows the principle of the Bayes theory (Example 2).

### BEST MODE FOR CARRYING OUT THE INVENTION

We sequenced the gene sequence in an upstream region from the translation initiation point to the 4120th nucleic-acid base and also the gene sequence in the coding region following to the translation initiation point in the glaucoma-responsible gene consisting of the nucleic-acid base sequence represented by SEQ ID No: 1 for glaucoma patients and non-patients. In the course of this work, we found a genetic polymorphism in the upstream region and the coding region of this gene, whose frequency differs between a patient group and a non-patient group. In addition, we found a fact that the glaucoma incidence differs in a statistically significant manner when compared with the incidence in an ordinary population on the basis of the presence or absence of this genetic polymorphism. The present invention is constituted of new findings described-above.

(Glaucoma-Related Gene)

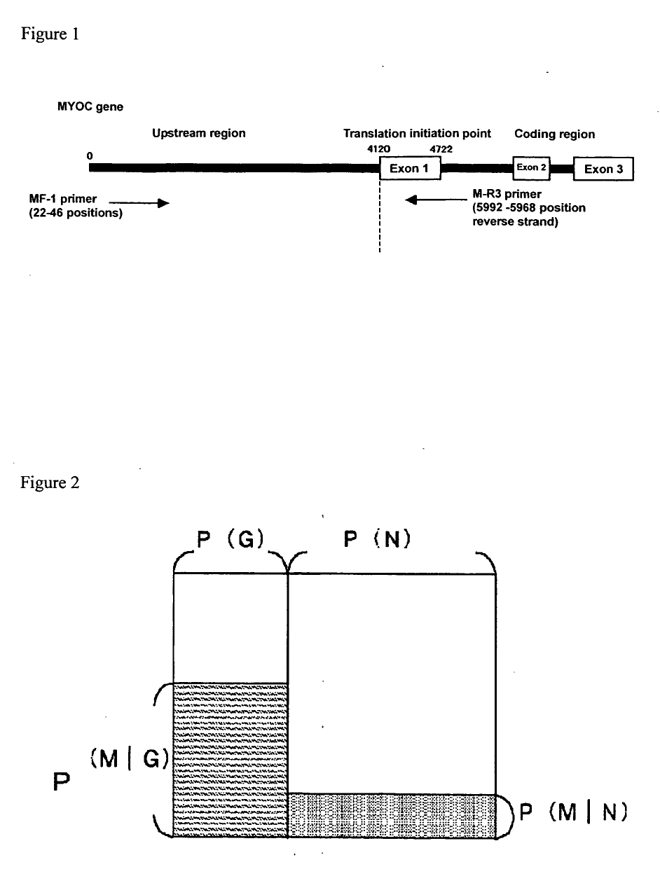

An inventive glaucoma-related gene may for example be a TIGR gene (trabecular reticulum-induced glucocorticoid responsive). This TIGR gene is known also as a MYOC gene. The structures and the sequences of the upstream and coding region of the MYOC gene are as shown in FIG. 1 and SEQ ID No: 1, and there are, for example, an upstream region with a promoter element and a coding region which encodes a protein, as well as other elements. The position of nucleic-acid base of MYOC gene is in accordance with the nucleic-acid base number defined in SEQ ID No: 1 (GenBank accession number: NT_{—}029874). The region which encodes this MYOC protein is formed from three exons. A region from position 1 to position 4120 in SEQ ID No: 1 is an upstream region, and a resion from position 4120 to position 4722-position represents Exon 1.

(Gene Variation)

A variation in an inventive glaucoma-related gene is a substitution of a nucleic-acid base in a particular position in an MYOC gene with a different nucleic-acid base, as well as deletion and/or insertion. Such a particular position is subjected for example to a substitution with a different base. Such a particular position refers a position selected from the 194-position, 199-position, position 324, position 1051, position 1084, position 1627, position 1685, position 1756, position 1853, position 2830, position 3371, position 4037 and/or position 4346 in the nucleic-acid base sequence represented by SEQ ID No: 1.

An inventive typical nucleic-acid base substitution in a particular position may for example be the C-to-A substitution at position 194; the A-to-C substitution at position 199; the G-to-A substitution at position 324; the C-to-T substitution at position 1051; the C-to-T substitution at position 1084; the T-to-C substitution at position 1627; the T-to-C substitution at position 1685; the C-to-T substitution at position 1756; the G-to-C substitution at position 1853; the G-to-A substitution at position 2830; the A-to-G substitution at position 3371; the G-to-A substitution at position 4037; and the G-to-A substitution at position 4346 in the nucleic-acid base sequence represented by SEQ ID No: 1.

Preferably, at least one substitution of nucleic-acid bases at position 199; position 324; position 1051; position 1084; position 1627; position 1685; position 1756; position 1853; position 2830 and position 3371 in the nucleic-acid base sequence represented by SEQ ID No: 1 is detected and subjected to a gene examination. More preferably, at least two substitutions of nucleic-acid bases at position 194; position 199; position 324; position 1051; position 1084; position 1627; position 1685; position 1756; position 1853; position 2830; position 3371; position 4037 and position 4346 in the nucleic-acid base sequence represented by SEQ ID No: 1 is detected.

(Examination Method)

The procedure in a method for examining a variation in the gene is not limited particularly as long as it can detect a particular variation of an inventive MYCO gene, and any of various methods which are known or will be available in future may be employed.

In order to examine an inventive variation in an MYOC gene in a subject, various methods for analyzing a nucleic-acid base sequence containing a position of such a variation can be employed. Such a method may for example be a southern hybridization method, dot hybridization method (see, for example, J. Mol. Biol., 98; 503-517 (1975)), dideoxy base sequencing method (Sanger method), various detection methods which combine DNA amplification technologies [for example, restriction fragment length polymorphism (RELF), PCR single strand higher structure polymorphism analysis (see, for example, Proc. Natl. Acad. Sci., U.S.A., 86:2766-2770 (1989)), PCR-specific sequence oligonucleotide method (SSO), allele-specific oligonucleotide method employing a PCR-SSO and a dot hybridization (see, for example, Nature, 324: 163-166 (1986))] and the like. As long as the position of the gene variation to be detected according to the invention is disclosed and specified, the variation can be detected using a method known in the art.

(Examination Sample Preparation)

In order to analyze an MYOC gene in a subject, an examination sample to be subjected to an inventive examination method is not limited particularly as long as it is a biological sample containing the MYOC gene in the subject. Such a biological sample may for example be a biological material tissue, surgically incised tissue, tissue isolated from a living body such as an oral mucosa tissue, as well as blood, serum, feces, injected semen, sputum, saliva, cerebrospinal fluid, hair and the like. An MYOC gene obtained by pelletizing a biological sample such as a tissue for example by a blender followed by extracting by a known gene extraction method such as a phenol/chloroform method can be used as an examination sample. The extracted MYOC gene may further be amplified and concentrated to give a sample.

A sample to be examined here may be a full-length DNA of an MYOC gene, or may be a DNA fragment (partial DNA). When a DNA fragment is subjected to the examination, it should contain a particular region which contains an upstream region and/or coding region of a MYOC gene and which contains a particular region involving a variation(s) in at least one position, preferably two or more position, more preferably 3 or more positions. Such a DNA fragment may not be limited particularly with regard to the base length, as long as it is one which can be utilized in detecting an inventive gene variation, i.e. one having a measurable base length of a subject DNA to be examined for the base substitution. The base length of such a DNA is usually 10 bases or more, preferably 20 bases or more, and generally, those having 100 to 1000, preferably 200 to 300 bases is selected.

A sample to be examined may be a DNA or a DNA transcription product. Typically, it may be a messenger RNA (mRNA) transcribed from a DNA, or its reverse transcription product cDNA, or a complementary DNA. Various procedures which can be employed in an inventive gene variation detecting method, including synthesis of a DNA or DNA fragment, enzyme treatment for a DNA cleavage, deletion, addition and binding, DNA isolation, purification, replication, selection as well as DNA fragment amplification, may be conducted by standard methods (see, for example, BUNSHIIDENGAKUJIKKENHO, KYORITSU SHUPPAN, published in 1983). A customary modification may also be conducted, if necessary.

The amplification of a nucleic acid for preparing a sample to be examined may be conducted for example by a PCR method or a modification thereof (see, for example, PCR Technology, Takara, published in 1990). In such a case, an oligonucleotide capable of being specifically hybridized with a part of a glaucoma-related gene, typically, an oligonucleotide possessing a primer function selected so that a desired DNA fragment having at least one particular position involved in the variation described above is amplified specifically, can be utilized.

(Primer Function-Possessing Oligonucleotide)

A primer function-possessing oligonucleotide may for example be 1) an oligonucleotide consisting of a nucleic-acid base sequence represented by any of SEQ ID Nos. 2 to 27; 2) a strand complementary with the oligonucleotide according to the above-mentioned 1); 3) an oligonucleotide capable of hybridizing under a stringent condition with the oligonucleotide according to the above-mentioned 1) or 2); 4) an oligonucleotide having a homology of about 60% with the oligonucleotide according to any one of the above-mentioned 1) to 3); and 5) an oligonucleotide having a nucleic-acid base sequence whose 1 or more nucleic-acid base(s) of oligonucleotides according to above-mentioned 1) to 4) was subjected to a variation such as a substitution, deletion, insertion or addition.

An oligonucleotide can be designed by a method known per se, and may for example be synthesized chemically. Alternatively, a naturally occurring nucleic acid can be cleaved for example by a restriction enzyme to alter into one having the nucleic-acid base sequence described above or can be ligated. Typically, the synthesis may be effected for example by using an oligonucleotide synthesizer (Applied Biosystems, Expedite Model 8909 DNA synthesizer). A method for synthesizing an oligonucleotide involving a variation such as a substitution, deletion, insertion of addition of one to several nucleic-acid base(s) may also be a method known per se. For example, a site-specific variation introduction method, gene homologous recombination method, primer extension method or polymerase chain reaction (PCR) method can be employed alone or in appropriate combination with each other, for example, in accordance for example with the methods described in Molecular Cloning: A Laboratory Mannual, 2nd ed., Sambrook et al., Cold Spring Harbor Laboratory Press, Cold Spring Harbor, N.Y., 1989; LABOMANUAL GENE ENGINEERING, ed by Muramatsu, Maruzene, 1988; PCR TECHNOLOGY, PRINCIPLE and APPLICATION OF DNA AMPLIFICATION, ed by Ehrlich, H. E., Stockton Press, 1989 and the like, with or without any modification, as well as the technology by Ulmer (Science (1983), 219:666).

A stringent hybridization condition may be any condition known commonly, such as the condition involving a hybridization at 42° C. overnight in a solution containing 50% formamide, 5×SSC (150 mM NaCl, 15 mM trisodium citrate), 50 mM sodium phosphate, pH 7.6, 5× Denhart's solution, 10% dextran sulfate and 20 μg/ml DNA, followed by a primary washing with 2×SSC/0.1% SDS at room temperature, followed by a secondary washing with 0.1×SSC/0.1% SDS at about 65° C.

(Detection of DNA Variation)

A variation in a DNA can be detected for example by sequencing the MYOC gene contained in a sample to be examined by Sanger method.

An oligonucleotide complementary with a single-stranded target MYOC gene is hybridized to the target MYOC gene and used as a primer to synthesis a complementary chain in the direction of 5′ to 3′ using a DNA polymerase. The oligonucleotide employed here may for example be an oligonucleotide described above (a primer function-possessing oligonucleotide), which is used as a primer.

In addition to 4 types of deoxyncleotide triphosphates (dNTPs), small amounts of dideoxyncleotide triphosphates (ddNTPs) are added as reaction substrates separately for respective nucleic-acid bases and the complementary chain is synthesized. ddNTP is a dNTP analogue formed as a result of the replacement of the —OH group at 3′-position of a deoxyribose with a —H group, and the incorporation of the ddNTP instead of the dNTP prevents any further synthesis of a complementary chain, thus enabling the synthesis of DNAs having various lengths. The addition for example of a chemiluminescence- or radioisotope (RI)-labeled primer or dNTP to a reaction system enables the labeling of a DNA to be synthesized, thus enabling the nucleic-acid base sequencing by subjecting the reaction products to an electrophoresis on a modified polyacrylamide gel.

A DNA polymerase employed in the Sanger method may for example be a klenow enzyme, T7 phage and thermophilic microorganism-derived DNA polymerase and the like. Commonly from these, the exonuclease activity was removed in a gene engineering manner. Initially, the Sanger method employed an intended gene as a single-stranded DNA, but currently a double-stranded plasmid is employed frequently as being alkali-modified directly.

A sequence reaction can be conducted by the Sanger method or a cycle sequence method. The cycle sequence method involves the Sanger method in combination with a PCR, and needs no process to convert a template DNA into a single strand, and employs a DNA, one primer, dNTPs, ddNTPs and a heat resistant DNA polymerase to be added to the reaction system. During the PCR reaction, the dNTPs are incorporated to arrest the chain elongation, as a result, allowing the DNAs having identical bases at their 3′-ends to be produced similarly to the Sanger method, The sequence reaction by an automatic sequencer may for example be a dye primer method using fluorescence-labeled primer, a dye terminator method using a fluorescence-labeled ddNTP and an internal-label method whose substrate dNTP is labeled.

(Examination Reagents and Examination Reagent Kits)

The invention also encompasses an examination reagent or examination reagent kit employed in a method for examining a glaucoma gene. The examination reagent may be any reagent employed in an inventive method including as a primer for amplifying a sample to be examined, a primer for nucleic-acid base sequencing of a sample to be examined, various polymerases, base substrates, labels and the like. The examination reagent kit may be any one which employs, as a kit, at least two of various reagents employed in an inventive method.

### EXAMPLES

The following Examples serve to further described the invention. However, they are not intended to restrict the invention in any way.

### Example 1

### DNA Analysis of MYOC Gene

(1) DNA Extraction

A blood donated from a subject to be examined was treated by a standard method and a DNA was extracted from a nucleating cell. The commercial DNA extraction kit “GenTORUKUN TM (for blood)” (Takara) was used and the DNA was extracted in accordance with the protocol attached thereto.

(2) Template DNA Amplification

The resultant DNA extract was employed as a template to amplify the MYOC gene by a PCR using a PCR amplification kit, trade name “LATaq” (Applied Biosystems). The amplification primers were an M-F1 (SEQ ID No:2) as a sense primer and an M-R3 (SEQ ID No:3) as an antisense primer. The M-F1 consists of a nucleic-acid base sequence represented in a region from position 22 to position 46 of SEQ ID No:1, while the M-R3 consists of the sequence complementary with the nucleic-acid base sequence represented in a region from position 5992 to position 5968. The reaction involved a heating at 94° C. for 1 minutes, followed by 30 cycles, each including at 94° C. for 30 seconds, at 60° C. for 30 seconds and then at 72° C. for 5 minutes and 30 seconds. The MYOC gene exon, translation initiation point and upstream structure, and the region amplified by the primers are in the relationship shown in FIG. 1.

(3) DNA Fragment Sequencing

The DNA fragments obtained by the PCR described above were subjected to an automatic DNA sequencer ABI Prism 3100 (Applied Biosystems) for sequencing the nucleic-acid base sequence of the DNA in accordance with the protocol attached thereto. The cycle sequence reaction was conducted here using the primers shown below.

The oligonucleotides represented by the following respective SEQ ID Nos. each consisting of a nucleic-acid base sequence contained in a region based on the nucleic-acid base sequence represented by SEQ ID No:1 or a complementary seqnence thereof were used as primers.

Forward Primers:

M-F1 Region from position 22 to position 46 (SEQ ID No: 2);
M-SF1 Region from position 372 to position 390 (SEQ ID No: 4);
M-SF2 Region from position 740 to position 759 (SEQ ID No: 5);
M-SF3 Region from position 1093 to position 1110 (SEQ ID No: 6);
M-SF4 Region from position 1456 to position 1475 (SEQ ID No: 7);
M-SF5 Region from position 1880 to position 1817 (SEQ ID No: 8);
M-SF6 Region from position 2148 to position 2165 (SEQ ID No: 9);
M-SF7 Region from position 2498 to position 2516 (SEQ ID No: 10);
M-SF8 Region from position 2857 to position 2875 (SEQ ID No: 11);
M-SF9 Region from position 3227 to position 3246 position (SEQ ID No: 12);
M-SF10 Region from position 3601 to position 3620 (SEQ ID No: 13);
M-SF11 Region form 3910 to position 3927 (SEQ ID No: 14);

Reverse Primers:
M-SR4 Resion from position 4730 to position 4712 complementary sequence (SEQ ID No: 15);
M-SR5 Resion from position 4337 to position 4319 complementary sequence (SEQ ID No: 16);
M-SR6 Resion from position 4022 to position 4003 complementary sequence (SEQ ID No: 17);
M-SR7 Resion from position 3712 to position 3695 complementary sequence (SEQ ID No: 18);
M-SR8 Resion from position 3379 to position 3360 complementary sequence (SEQ ID No: 19);
M-SR9 Resion from position 2950 to position 2933 complementary sequence (SEQ ID No: 20);
M-SR10 Resion from position 2593 to position 2575 complementary sequence (SEQ ID No: 21);
M-SR11 Resion from position 2259 to position 2241 complementary sequence (SEQ ID No: 22);
M-SR12 Resion from position 1950 to position 1933 complementary sequence (SEQ ID No: 23);
M-SR13 Resion from position 1556 to position 1538 complementary sequence (SEQ ID No: 24);
M-SR14 Resion from position 1170 to position 1153 complementary sequence (SEQ ID No: 25);
M-SR15 Region from position 824 to position 807 complementary sequence (SEQ ID No: 26);
M-SR16 Resion from position 470 to position 453 complementary sequence (SEQ ID No: 27).

Among the primers listed above, M-F1 through M-SF11 were used for sequencing the forward strands, while M-SR4 through M-SR1 6 were used for sequencing the reverse strands.

(4) DNA Fragment Ligation and MYOC Gene Nucleic-Acid Base Sequence

The sequence of the DNA fragment of individual blood donor was ligated using a Phred/Phrap software (Washington University, USA) and one nucleic-acid base sequence per blood donor was obtained.

The blood samples obtained from a control group of 67 non-patient volunteers were treated in accordance with the above-mentioned procedure, and the nucleic-acid base sequence (SEQ ID No: 1) of the MYOC gene which was predominant in the non-patient group was determined.

### Example 2

(1) MYOC Gene Nucleic-Acid Base Sequence Polymorphism Analysis 1

The blood samples obtained from 88 patients each diagnosed to have an open-angle glaucoma by a medical center were treated in accordance with the procedure of the above-mentioned Example and the nucleic-acid base sequence of the MYOC gene of each patient was analyzed and compared with the nucleic-acid base sequence of the non-patient group.

The results are shown in Table 1. The first row in Table 1 represents the position of the nucleic-acid base sequence of the MYOC gene represented by SEQ ID No: 1, the second row represents the base predominant in the non-patient group in each position, the third row represents the frequency of the variation in each base position in the non-patient group, the fourth row represents the frequency of the variation in each base position in the patient group and the fifth row represents the change in the nucleic-acid base detected as a variation.

As a result, the variation at nucleic-acid base positions of position 324, position 4037 and position 4346 was found at a frequency of about 3% in the non-patient group, while the frequency of the variation in the patient group was 6.8 to 10.2%. In other positions, there was no variation found in the non-patient group, but the variation was found at a frequency of about 1 to 3.4% in the patient group.

TABLE 1

| | Base position | | | | | | | | | | | | |
|---|---|---|---|---|---|---|---|---|---|---|---|---|---|
| | 194 | 199 | 324 | 1051 | 1084 | 1627 | 1685 | 1756 | 1853 | 2830 | 3371 | 4037 | 4346 |
| Nucleic-acid base | C | A | G | C | C | T | T | C | G | G | A | G | G |
| Non-patient group | 0.0% | 0.0% | 3.0% | 0.0% | 0.0% | 0.0% | 0.0% | 0.0% | 0.0% | 0.0% | 0.0% | 3.0% | 3.0% |
| Patient group | 3.4% | 1.1% | 6.8% | 2.3% | 3.4% | 3.4% | 2.3% | 2.3% | 2.3% | 1.1% | 1.1% | 10.2% | 10.2% |
| Variation | C→A | A→C | G→A | C→T | C→T | T→C | T→C | C→T | G→C | G→A | A→G | G→A | G→A |

(2) MYOC Gene Nucleic-Acid Base Sequence Polymorphism Analysis 2

11 Patients and 2 non-patients having variations in the nucleic-acid bases at position 4037 and position 4346 were also examined for the variations at other positions.

The results are shown in Table 2. The first row of Table 2 represents the nucleic-acid base position, and the second row or later represent the presence or absence of the variation in individual subject.

As a result, the non-patient group exhibited no variations in the base positions other than the positions 4037 and 4346, while the patient group included a subgroup with the variations in the 194, 1084-and position 1627s and a subgroup with the variations in the 1051-, 1685-, 1756- and position 1853s, or a subgroup without variations.

TABLE 2

| | Base position | | | | | | | | | | | | |
|---|---|---|---|---|---|---|---|---|---|---|---|---|---|
| | 194 | 199 | 324 | 1051 | 1084 | 1627 | 1685 | 1756 | 1853 | 2830 | 3371 | 4037 | 4346 |
| Patient 1 | * | | | | * | * | | | | | | * | * |
| Patient 2 | * | | | | * | * | | | | | | * | * |
| Patient 3 | * | | | | * | * | | | | | | * | * |
| Patient 4 | | | | | | | | | | | | * | * |
| Patient 5 | | | | | | | | | | | | * | * |
| Patient 6 | | | | | | | | | | | | * | * |
| Patient 7 | | | | | | | | | | | | * | * |
| Patient 8 | | | | | | | | | | | | * | * |
| Patient 9 | | | | | | | | | | | | * | * |
| Patient 10 | | | | * | | | * | * | * | | | | |
| Patient 11 | | | | * | | | * | * | * | | | | |
| Non-patient 1 | | | | | | | | | | | | * | * |
| Non-patient 2 | | | | | | | | | | | | * | * |

(3) Risk Judgment

According to the Bayes theory, the risk of developing glaucoma when there is a variation in the MYOC gene sequence is predicted.

The probability of developing glaucoma in future of a in a given subject is judged based on the incidence in a epidemiologically categorized general population, if there is no information beforehand. When this incidence is designated as P(G) and the probability of no development of glaucoma is designated as P(N), then there is a relationship of P(N)=1−P(G).

On the other hand, the case where there is a single or multiple variation in the MYOC gene sequence is designated as M, and the conditional probability that a subject having M will undergo the development of glaucoma is designated as P(G|M). If P(G|M)>P(G), then the probability that the variation M-possessing subject will undergo the development of glaucoma is higher than that in the general population, resulting in a judgment of a high risk subject.

The conditional probability P(G|M) is calculated as follows.

The probability of the possession of M in the glaucoma patient group is designated as P(M|G), while the probability of the possession of M in the non-patient group is designated as P(M|N). Then the P(M|N) is represented by Equation 1 according to the Bayes theory (FIG. 2). The value of P(G) may employ a value of 3.56%, which was reported to be the glaucoma patient ratio in the population of 40 years old or older based on the nationwide epidemiological investigation conducted over the period from 1988 to 1989 by Japan Ophthalmologists Association. On the other hand, the probability of the variation in a nucleic-acid base in the MYOC gene in each position in the glaucoma patient and non-patient groups can be extrapolated into Equation 1, P(M|G) value and P(M|N) value, respectively.

$$P ( G \vert M ) = \frac{P ( G ) \times P ( M \vert G )}{P ( G ) \times P ( M \vert G ) + P ( N ) \times P ( M \vert N )} \qquad ( Equation 1 )$$

Using the equation shown above to calculate P(G|M) for each variation, the ratio of 194-position=28.1, 199-position=28.1, position 324=2.184, position 1051=28.1, position1084=28.1, position1627=28.1, position 1685=28.1, position 1756=28.1, position 1853=28.1, position 2830=28.1, position 3371=28.1, position 4037=3.2, position 4346=3.2, exhibiting a higher value of P(G|M) than P(G), which revealed that the possession of a variance in each of these positions reflects a high risk group of the glaucoma. Accordingly, the detection of the variations in the positions in the gene described above is effective in predicting the risk of developing open-angle glaucoma.

(4) Effective Examination For Risk Judgment

Then, the positions of the variations in the MYOC gene for ensuring an effective risk judgment were analyzed, and the results are shown in Table 2/

Since the probability in the patients having the a variation at position 4037 or position 4346 is about 10%, the risk of developing glaucoma in an individual having a variation in these positions can be judged first. On the other hand, any individual having a variation in these positions has the variations in the both positions. Accordingly, the variation of either one of the position 4037 and position 4346 is detected and employed as an index for judging the glaucoma developmental risk.

Nevertheless, the probabilities of the patients having a variation at position 4037 or position 4346 are about 3% respectively, even in the non-patient group. On the other hand, among the patients having the variation at position 4037 and position 4346, the ones having variations in all of the 194-, 1084-and position 1627s were all the patients (Patient 1, Patient 2, Patient 3). Therefore, if a variation in at least one position of positions 194, 1084 and 1627 is detected in addition to the variation of either one of the position 4037 and position 4346 and used as an index, the pseudo-positive judgment can be avoided in predicting the glaucoma onset.

Similarly, the ones having variations in all of the 1084-, 1685-, 1756-and position 1853s were all the patients (Patient 10, Patient 11). Therefore, if the variation in at least one position of the 1084-, 1685-, 1756-and position 1853s is detected and used as an index, the pseudo-positive judgment can be avoided in predicting the development of glaucoma.

### INDUSTRIAL APPLICABILITY

As described above, the information with regard to the gene variation according to the invention is useful in predicting developing in future of glaucoma. Especially, if the development of open-angle glaucoma can be predicted by detecting the MYOC gene variation using an inventive gene examination method, the prevention of the development at a stage before the development or an early treatment is possible.

1. A method for examining a gene comprising detecting a variation in nucleic-acid bases in at least two or more positions within a gene region containing a glaucoma-related gene coding region and/or an upstream region and predicting any future development of glaucoma using said variation as an index.

2. The method according to claim 1 wherein the glaucoma-related gene is a myocilin (MYOC) gene.

3. The method according to claim 1 wherein the gene region is the nucleic-acid base sequence represented by SEQ ID No:1.

4. The method according to claim 1 wherein the variation in a base is a substitution, deletion and/or insertion.

5. The method according to claim 3 which detects any of the group consisting of, in the nucleic-acid base sequence represented by SEQ ID No:1, the C-to-A substitution at position 194; the A-to-C substitution at position 199; the G-to-A substitution at position 324; the C-to-T substitution at position 1051; the C-to-T substitution at position 1084; the T-to-C substitution at position 1627; the T-to-C substitution at position 1685; the C-to-T substitution at position 1756; the G-to-C substitution at position 1853; the G-to-A substitution at position 2830; the A-to-G substitution at position 3371; the G-to-A substitution at position 4037; and the G-to-A substitution at position 4346.

6. The method according to claim 3 which detects at least two or more simultaneous substitutions selected from the group consisting of, in the nucleic-acid base sequence represented by SEQ ID No:1, the C-to-A substitution at position 194; the C-to-T substitution at position 1084; the T-to-C substitution at position 1627; the G-to-A substitution at position 4037; and the G-to-A substitution at position 4346.

7. The method according to claim 3 which detects at least two or more simultaneous substitutions selected from the group consisting of, in the nucleic-acid base sequence represented by SEQ ID No:1, the C-to-T substitution at position 1051; the T-to-C substitution at position 1685; the C-to-T substitution at position 1756; and the G-to-C substitution at position 1853.

8. A method for examining a gene comprising detecting at least one substitution of the group consisting of, in the nucleic-acid base sequence represented by SEQ ID No: 1, the A-to-C substitution at position 199; the G-to-A substitution at position 324; the C-to-T substitution at position 1051; the C-to-T substitution at position 1084; the T-to-C substitution at position 1627; the T-to-C substitution at position 1685; the C-to-T substitution at position 1756; the G-to-C substitution at position 1853; the G-to-A substitution at position 2830; and the A-to-G substitution at position 3371 and predicting any future development of glaucoma using said variation as an index.

9. The method according to claim 1 wherein the glaucoma is primary open-angle glaucoma and/or normal tension glaucoma.

10. The method according to claim 1 wherein the variation is detected using an oligonucleotide capable of specifically forming a hybrid with a part of a gene region containing a glaucoma-related gene coding region and/or an upstream region.

11. A primer function-possessing oligonucleotide wherein the oligonucleotide, which is capable of specifically forming a hybrid with a part of a gene region containing a glaucoma-related gene coding region and/or an upstream region, is at least one or more selected from the group consisting of:
1) an oligonucleotide consisting of a nucleic-acid base sequence represented by any of SEQ ID Nos. 2 to 27;
2) a strand complementary with the oligonucleotide according to the above-mentioned 1);
3) an oligonucleotide capable of hybridizing under a stringent condition with the oligonucleotide according to the above-mentioned 1) or 2);
4) an oligonucleotide having a homology of about 60% with the oligonucleotide according to any one of the above-mentioned 1) to 3);
5) an oligonucleotide having a nucleic-acid base sequence whose 1 or more base of oligonucleotides according to the above-mentioned 1) to 4) was subjected to a variation such as a substitution, deletion, insertion or addition.

12. The method according to claim 1 comprising performing a nucleic acid amplification process using at least one oligonucleotide selected from the oligonucleotides according to claim 11.

13. An examination reagent or examination reagent kit comprising a reagent employed in the examination method according to claim 12.

14. The method according to claim 2 wherein the gene region is the nucleic-acid base sequence represented by SEQ ID No:1.

15. The method according to claim 2 wherein the variation in a base is a substitution, deletion and/or insertion.

16. The method according to claim 3 wherein the variation in a base is a substitution, deletion and/or insertion.

17. The method according to claim 2 wherein the glaucoma is primary open-angle glaucoma and/or normal tension glaucoma.

18. The method according to claim 2 wherein the variation is detected using an oligonucleotide capable of specifically forming a hybrid with a part of a gene region containing a glaucoma-related gene coding region and/or an upstream region.

19. The method according to claim 3 wherein the glaucoma is primary open-angle glaucoma and/or normal tension glaucoma.

20. The method according to claim 3 wherein the variation is detected using an oligonucleotide capable of specifically forming a hybrid with a part of a gene region containing a glaucoma-related gene coding region and/or an upstream region.


SEQUENCE LISTING (27 sequences)

SEQ ID NO: 1 - DNA, 6000 bp, Homo sapiens
gctccacaggaagtctccccactctagacttctgcatcacgatgttacagccagaagctccgtgagggtgagggtctgtgtcttacacctacctgtatgctctacacctgagctcactgcaacctctgcctcccaggttcaagcaattctcctgtctcagcctcccgcgtagctgggactacaggcgcacgcccggctaatttttgtattgttagtagagatggggtttcaccatattagcccggctggtcttgaactcctgacctcaggtgatccacccacctcagcctcctaaagtgctgggattacaggcatgagtcaccgcgcccggccaagggtcagtgtttaataaggaataacttgaatggtttactaaaccaacagggaaacagacaaaagctgtgataatttcagggattcttgggatggggaatggtgccatgagctgcctgcctagtcccagaccactggtcctcatcactttcttccctcatcctcattttcaggctaagttaccattttattcaccatgcttttgtggtaagcctccacatcgttactgaaataagagtatacataaactagttccatttggggccatctgtgtgtgtgtataggggaggagggcataccccagagactccttgaagcccccggcagaggtttcctctccagctgggggagccctgcaagcacccggggtcctgggtgtcctgagcaacctgccagcccgtgccactggttgttttgttatcactctctagggacctgttgctttctatttctgtgtgactcgttcattcatccaggcattcattgacaatttattgagtacttatatctgccagacaccagagacaaaatggtgagcaaagcagtcactgccctaccttcgtggaggtgacagtttctcatggaagacgtgcagaagaaaattaatagccagccaacttaaacccagtgctgaaagaaaggaaataaacaccatcttgaagaattgtgcgcagcatcccttaacaaggccacctccctagcgccccctgctgcctccatcgtgcccggaggcccccaagcccgagtcttccaagcctcctcctccatcagtcacagcgctgcagctggcctgcctcgcttcccgtgaatcgtcctggtgcatctgagctggagactccttggctccaggctccagaaaggaaatggagagggaaactagtctaacggagaatctggaggggacagtgtttcctcagagggaaaggggcctccacgtccaggagaattccaggaggtggggactgcagggagtggggacgctggggctgagcgggtgctgaaaggcaggaaggtgaaaagggcaaggctgaagctgcccagatgttcagtgttgttcacggggctgggagttttccgttgcttcctgtgagcctttttatcttttctctgcttggaggagaagaagtctatttcatgaagggatgcagtttcataaagtcagctgttaaaattccagggtgtgcatgggttttccttcacgaaggcctttatttaatgggaatataggaagcgagctcatttcctaggccgttaattcacggaagaagtgactggagtcttttctttcatgtcttctgggcaactactcagccctgtggtggacttggcttatgcaagacggtcgaaaaccttggaatcaggagactcggttttctttctggttctgccattggttggctgtgcgaccgtgggcaagtgtctctccttccctgggccatagtcttctctgctataaagacccttgcagctctcgtgttctgtgaacacttccctgtgattctctgtgaggggggatgttgagaggggaaggaggcagagctggagcagctgagccacaggggaggtggagggggacaggaaggcaggcagaagctgggtgctccatcagtcctcactgatcacgtcagactccaggaccgagagccacaatgcttcaggaaagctcaatgaacccaacagccacattttccttccctaagcatagacaatggcatttgccaataaccaaaaagaatgcagagactaactggtggtagcttttgcctggcattcaaaaactgggccagagcaagtggaaaatgccagagattgttaaacttttcaccctgaccagcaccccacgcagctcagcagtgactgctgacagcacggagtgacctgcagcgcaggggaggagaagaaaaagagagggatagtgtatgagcaagaaagacagattcattcaagggcagtgggaattgaccacagggattatagtccacgtgatcctgggttctaggaggcagggctatattgtggggggaaaaaatcagttcaagggaagtcgggagacctgatttctaatactatatttttcctttacaagctgagtaattctgagcaagtcacaaggtagtaactgaggctgtaagattacttagtttctccttattaggaactctttttctctgtggagttagcagcacaagggcaatcccgtttcttttaacaggaagaaaacattcctaagagtaaagccaaacagattcaagcctaggtcttgctgactatatgattggttttttgaaaaatcatttcagcgatgtttactatctgattcagaaaatgagactagtaccctttggtcagctgtaaacaaacacccatttgtaaatgtctcaagttcaggcttaactgcagaaccaatcaaataagaatagaatctttagagcaaactgtgtttctccactctggaggtgagtctgccagggcagtttggaaatatttacttcacaagtattgacactgttgttggtattaacaacataaagttgctcaaaggcaatcattatttcaagtggcttaaagttacttctgacagttttggtatatttattggctattgccatttgctttttgttttttctctttgggtttattaatgtaaagcagggattattaacctacagtccagaaagcctgtgaatttgaatgaggaaaaaattacatttttgtttttaccaccttctaactaaatttaacattttattccattgcgaatagagccataaactcaaagtggtaataacagtacctgtgattttgtcattaccaatagaaatcacagacattttatactatattacagttgttgcagatacgttgtaagtgaaatatttatactcaaaactactttgaaattagacctcctgctggatcttgtttttaacatattaataaaacatgtttaaaattttgatattttgataatcatatttcattatcatttgtttcctttgtaatctatattttatatatttgaaaacatctttctgagaagagttccccagatttcaccaatgaggttcttggcatgcacacacacagagtaagaactgatttagaggctaacattgacattggtgcctgagatgcaagactgaaattagaaagttctcccaaagatacacagttgttttaaagctaggggtgaggggggaaatctgccgcttctataggaatgctctccctggagcctggtagggtgctgtccttgtgttctggctggctgttatttttctctgtccctgctacgtcttaaaggacttgtttggatctccagttcctagcatagtgcctggcacagtgcaggttctcaatgagtttgcagagtgaatggaaatataaactagaaatatatccttgttgaaatcagcacaccagtagtcctggtgtaagtgtgtgtacgtgtgtgtgtgtgtgtgtgtgtgtgtaaaaccaggtggagatataggaactattattggggtatgggtgcataaattgggatgttctttttaaaaagaaactccaaacagacttctggaaggttattttctaagaatcttgctggcagcgtgaaggcaacccccctgtgcacagccccacccagcctcacgtggccacctctgtcttcccccatgaagggctggctccccagtatatataaacctctctggagctcgggcatgagccagcaaggccacccatccaggcacctctcagcacagcagagctttccagaggaagcctcaccaagcctctgcaatgaggttcttctgtgcacgttgctgcagctttgggcctgagatgccagctgtccagctgctgcttctggcctgcctggtgtgggatgtgggggccaggacagctcagctcaggaaggccaatgaccagagtggccgatgccagtataccttcagtgtggccagtcccaatgaatccagctgcccagagcagagccaggccatgtcagtcatccataacttacagagagacagcagcacccaacgcttagacctggaggccaccaaagctcgactcagctccctggagagcctcctccaccaattgaccttggaccaggctgccaggccccaggagacccaggaggggctgcagagggagctgggcaccctgaggcgggagcgggaccagctggaaacccaaaccagagagttggagactgcctacagcaacctcctccgagacaagtcagttctggaggaagagaagaagcgactaaggcaagaaaatgagaatctggccaggaggttggaaagcagcagccaggaggtagcaaggctgagaaggggccagtgtccccagacccgagacactgctcgggctgtgccaccaggctccagagaaggtaagaatgcagagtggggggactctgagttcagcaggtgatatggctcgtagtgacctgctacaggcgctccaggcctccctgcctgccctttctcctagagactgcacagctagcacaagacagatgaattaaggaaagcacagcgatcaccttcaagtattactagtaatttagctcctgagagcttcatttagattagtggttcagagttcttgtgcccctccatgtcagttttcacagtccatagcaaaaggagaaataaaaggaccgggtgagatgtgtctgcatatgagcagtagaaagttgtcaattgtcccttttgaaaaactatccttttttgaacctttgctcagattgttatttgtaccttttgatgttaaaatgacctttatttatgaaattacaatagatttgggaaatgataataagtggtaagtttttgtttatttttaaatgttcttccctggcaaaataaagagatggcacctctctgtcagttttcttaatatgttgttctgaaagttttcttactcagtccaatctgagaacctctgcttttaagtcatcagacaaattcttgagatggctttttctgagaggctcttctgttcatcctggtcccttcttgcctaaaggtgagtctgtgtgtgtgtggggggggtgcgggggtgaggtgttgggggaggtcttcttattagctgggaagatggtatttgtgtcactttttgtgaaagtgggctcccaaatattccctgttgaggaagtgttctaatcatgaggaaataagcaagcaaatccagttgttggacaattagtttggactggtcaaagatgtcagtgccaaggaagaaagaaaaaaggggtggggaagggcttgttctatattaaagagactaaagaaatgtgttaaccaaatgtagtgcatgagtcttgattggtgtcttcatccaagggggaaaaaggctatgaggaacaggtttgggataactgaggcaatttgactgctcattattatgttactgtattaatgttcagtttcttggtgagataatgatactgtggttgcgaaggataaaatctttgttctatggagatacatgcttaagtacccagggtgaggcgtcaggatgtctgcaatttgctctcaaatggttgaagaaagactgcaaatatatagataatgagagaaagaaaggtaaaacaactgtggcaaaatattaataactggtgaattacaaactggtgaatctaagtatatggggagcttattgtac

SEQ ID NO: 2 - DNA, 25 bp, Artificial Sequence
  Designed DNA based on MYOC gene
ctctagacttctgcatcacgatgtt

SEQ ID NO: 3 - DNA, 25 bp, Artificial Sequence
  Designed DNA based on MYOC gene
agctccccatatacttagattcacc

SEQ ID NO: 4 - DNA, 19 bp, Artificial Sequence
  Desiend DNA based on MYOC gene
actaaaccaacagggaaac

SEQ ID NO: 5 - DNA, 20 bp, Artificial Sequence
  Designed DNA based on MYOC gene
tggttgttttgttatcactc

SEQ ID NO: 6 - DNA, 18 bp, Artificial Sequence
  Designed DNA based on MYOC gene
ctcctccatcagtcacag

SEQ ID NO: 7 - DNA, 20 bp, Artificial Sequence
  Deisigned DNA based on MYOC gene
gaagtctatttcatgaaggg

SEQ ID NO: 8 - DNA, 18 bp, Artificial Sequence
  Designed DNA based on MYOC gene
agctctcgtgttctgtga

SEQ ID NO: 9 - DNA, 18 bp, Artificial Sequence
  Designed DNA based on MYOC gene
aaacttttcaccctgacc

SEQ ID NO: 10 - DNA, 19 bp, Artificial Sequence
  Designed DNA based on MYOC gene
ttctctgtggagttagcag

SEQ ID NO: 11 - DNA, 19 bp, Artificial Sequence
  Designed DNA based on MYOC gene
acataaagttgctcaaagg

SEQ ID NO: 12 - DNA, 20 bp, Artificial Sequence
  Designed DNA based on MYOC gene
ctttgaaattagacctcctg

SEQ ID NO: 13 - DNA, 20 bp, Artificial Sequence
  Designed DNA based on MYOC gene
gctgttatttttctctgtcc

SEQ ID NO: 14 - DNA, 18 bp, Artificial Sequence
  Designed DNA based on MYOC gene
ctaagaatcttgctggca

SEQ ID NO: 15 - DNA, 19 bp, Artificial Sequence
  Designed DNA based on MYOC gene
ttcttaccttctctggagc

SEQ ID NO: 16 - DNA, 19 bp, Artificial Sequence
  Designed DNA based on MYOC gene
ttatggatgactgacatgg

SEQ ID NO: 17 - DNA, 20 bp, Artificial Sequence
  Designed DNA based on MYOC gene
tttatatatactggggagcc

SEQ ID NO: 18 - DNA, 18 bp, Artificial Sequence
  Designed DNA based on MYOC gene
ccattcactctgcaaact

SEQ ID NO: 19 - DNA, 20 bp, Artificial Sequence
  Designed DNA based on MYOC gene
ggaactcttctcagaaagat

SEQ ID NO: 20 - DNA, 18 bp, Artificial Sequence
  Designed DNA based on MYOC gene
aaaagcaaatggcaatag

SEQ ID NO: 21 - DNA, 19 bp, Artificial Sequence
  Designed DNA based on MYOC gene
gacctaggcttgaatctgt

SEQ ID NO: 22 - DNA, 19 bp, Artificial Sequence
  Designed DNA based on MYOC gene
tgctcatacactatccctc

SEQ ID NO: 23 - DNA, 18 bp, Artificial Sequence
  Desigend DNA based on MYOC gene
agtgaggactgatggagc

SEQ ID NO: 24 - DNA, 19 bp, Artificial Sequence
  Designed DNA based on MYOC gene
attcccattaaataaaggc

SEQ ID NO: 25 - DNA, 18 bp, Artificial Sequence
  Designed DNA based on MYOC gene
agtctccagctcagatgc

SEQ ID NO: 26 - DNA, 18 bp, Artificial Sequence
  Designed DNA based on MYOC gene
attgtcaatgaatgcctg

SEQ ID NO: 27 - DNA, 18 bp, Artificial Sequence
  Designed DNA based on MYOC gene
cagtggtctgggactagg